(19) United States
(12) Patent Application Publication
(10) Pub. No.: US 2025/0312059 A1
(43) Pub. Date: Oct. 9, 2025

(54) ATRAUMATIC MALE CIRCUMCISION APPARATUS WITH LATERAL STABILIZATION LOCKING FEATURES

(71) Applicant: SafeCirc, LLC, Wakefield, RI (US)

(72) Inventors: David Tomlinson, Wakefield, RI (US); Richard Dixon, Bountiful, UT (US)

(21) Appl. No.: 18/860,555

(22) PCT Filed: Apr. 28, 2023
(86) PCT No.: PCT/US2023/066397

§ 371 Date: Oct. 25, 2024

Related U.S. Application Data
(60) Provisional application No. 63/336,006, filed on Apr. 28, 2022.

(51) Int. Cl. A61B 17/326

(52) U.S. Cl. CPC A61B 17/326

(57) ABSTRACT

A device for performing a male circumcision includes a ring component and a foreskin holder. The ring component includes a ring portion and a shaft defining a longitudinal axis and extending proximally from the ring portion. The foreskin holder couples to the ring component and is configured to move axially along the shaft and includes a ring portion, wherein distal movement of the foreskin holder clamps a foreskin between the ring portions. In some embodiments, the shaft includes a longitudinal groove and the foreskin holder includes a lateral tab that engages with the longitudinal groove to inhibit lateral movement of the foreskin holder with respect to the ring component. In some embodiments, the shaft includes a laterally-projecting lip and the foreskin holder includes an arm that engages with the laterally-extending lip to lock the axial position of the foreskin holder.

### FIELD OF THE DISCLOSURE

The present disclosure generally relates to a device for holding foreskin in place during male circumcision.

### BRIEF DESCRIPTION OF THE DRAWINGS

FIG. 1 is a perspective view of an example foreskin holder.

FIG. 2 is a partial front view of a portion of the foreskin holder of FIG. 1.

FIG. 3 a top view of the foreskin holder of FIG. 1.

FIG. 4 is a perspective view of a portion of a ring component that may find use with the foreskin holder of FIGS. 1-3.

FIGS. 5A and 5B illustrate various stages of an example foreskin holder and ring component.

FIGS. 6A and 6B illustrate various stages of an example foreskin holder and ring component.

FIG. 7A is a perspective view of one embodiment of the present invention shown in the up position or disengaged;

FIG. 7B is a perspective view of one embodiment of the present invention shown in the down or engaged position;

FIG. 8 is a perspective view of an example foreskin holder.

FIGS. 9A-B are side views of the foreskin holder of FIG. 8.

FIG. 9C is a bottom view of the foreskin holder of FIG. 8.

FIGS. 10A-C illustrate the assembly of the foreskin holder of FIG. 8 on to an example embodiment of the ring component.

FIGS. 11-14 are pictorial views of the ring component being inserted into the foreskin of a penis.

FIGS. 15A-C illustrate the positioning of foreskin between the foreskin holder and the ring component of FIGS. 10A-C.

FIG. 16 includes various views illustrating use of an open ring of the present disclosure with a clamping-cutting device.

### DETAILED DESCRIPTION

U.S. Pat. No. 8,353,918 (“'918 Patent”), which is hereby incorporated by reference in its entirety, discloses a method of using a disposable neonatal male circumcision device that secures the foreskin of the penis in a precise location, applies radially circumferential clamping, and delivers a longitudinal circumferential cutting device along the path precisely controlled by the device itself, not the operator, ensuring the incision to the clamped foreskin is made in the precise location, independent of the operator.

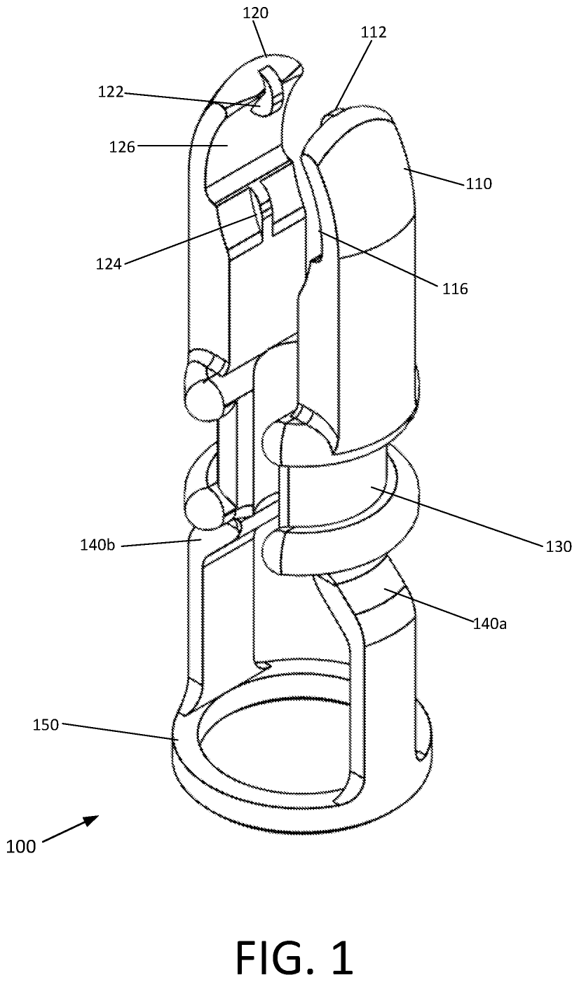

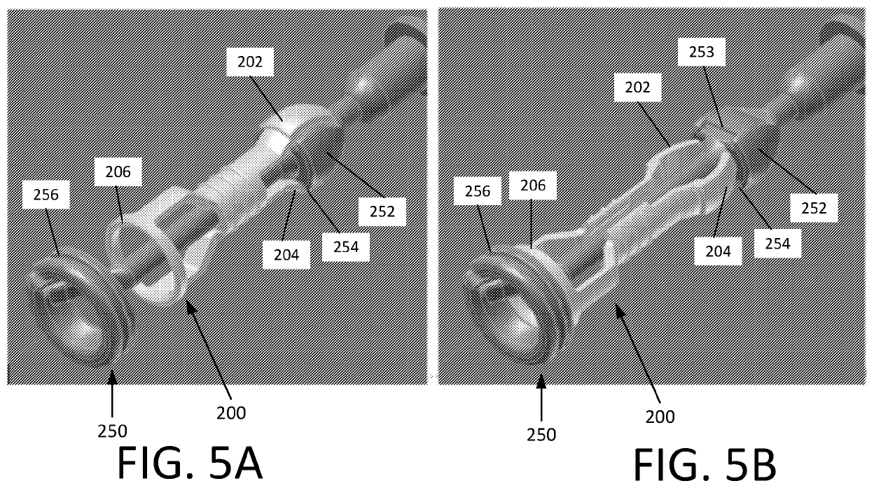

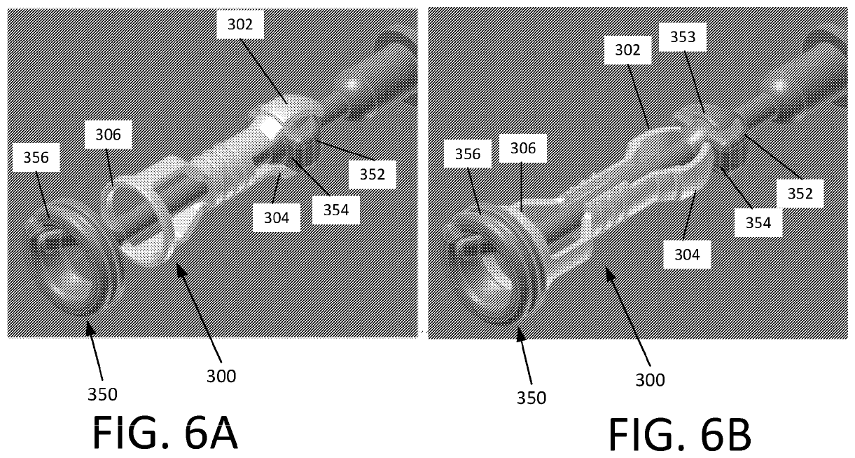

The instant disclosure includes improved devices and methods for performing male circumcision, for example as improvements to the device of the '918 Patent. In a first example discussed below with respect to FIGS. 1-4, a foreskin holder may include tabs that interact with grooves on a ring component to restrict lateral (e.g., rotational) movement of the foreskin holder relative to the ring component. The tabs may correspond to and interact with grooves on a position adjustor of the ring component, such that the tabs of the foreskin holder move along (e.g., within) the grooves as the foreskin holder moves relative to the position adjustor. The restriction on lateral movement enabled by the tabs and grooves may result in a more stable clamp between the foreskin holder and the ring component. In a second example discussed below with respect to FIGS. 5A-6B, the position adjustor may include a locking ridge that interacts with arms of the foreskin holder to secure the foreskin holder in a clamped position.

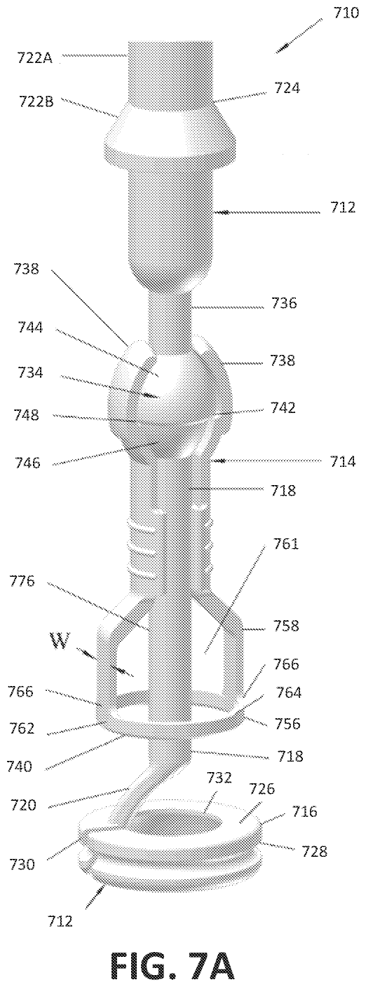

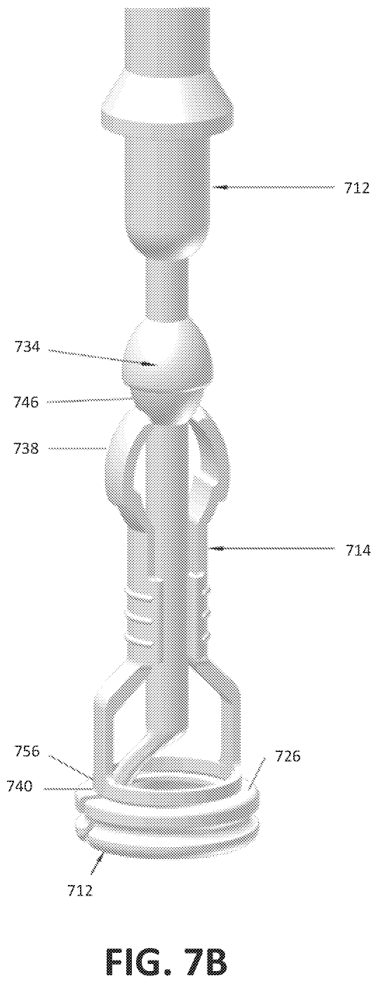

FIGS. 7A-16 illustrate embodiments of devices for performing male circumcision operations, showing broader structure and processes for such an operation. It will be appreciated that the lateral and axial stability components illustrated in and described with respect to FIGS. 1-6B can readily be incorporated into or otherwise combined with the broader structure and processes of FIGS. 7A-16 to improve upon the structure and processes of FIGS. 7A-16.

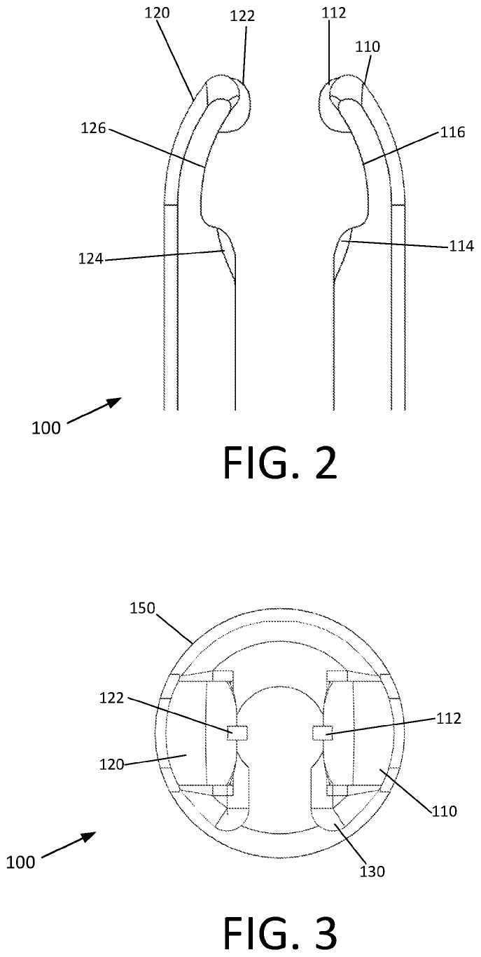

Referring to the drawings, wherein like reference numerals refer to the same or similar features in the various views, FIG. 1 is a perspective view of an example foreskin holder 100. The foreskin holder 100 may include a first arm 110, a second arm 120, a C-shaped sleeve 130, a first support 140a and a second support 140b (collectively “supports 140”), and a ring portion 150. FIG. 2 is a partial front view of the foreskin holder 100 to show detail of the first arm 110 and the second arm 120. FIG. 3 is a top view of the foreskin holder 100. The C-shaped sleeve 130 may be structurally similar to the “C-shaped sleeve 160” of the '918 Patent, for example, including the associated broader structure of which the C-shaped sleeve 130 may be a part. The supports 140 may be structurally similar to the “pair of support arms 158” of the '918 Patent, for example, including the associated broader structure of which the supports 140 may be a part. The ring portion 150 may be structurally similar to the “ring portion 156” of the '918 Patent, for example, including the associated broader structure of which the ring portion 150 may be a part. Relative to the structures disclosed in the '918 Patent, the foreskin holder 100 may include tabs 112, 114, 122, 124 for interacting with corresponding grooves to provide improved lateral stability, as discussed below.

Referring to FIGS. 1-3, the first arm 110 may include a first upper tab 112, a first lower tab 114, and a first concave portion 116 that extends from the first upper tab 112 to the first lower tab 114. Similarly but on an opposite side of the foreskin holder 100, the second arm 120 may include a second upper tab 122, a second lower tab 124, and a second concave portion 126 that extends from the second upper tab 122 to the second lower tab 124, such that that first arm 110 and the second arm 120 may be identical and mirrored.

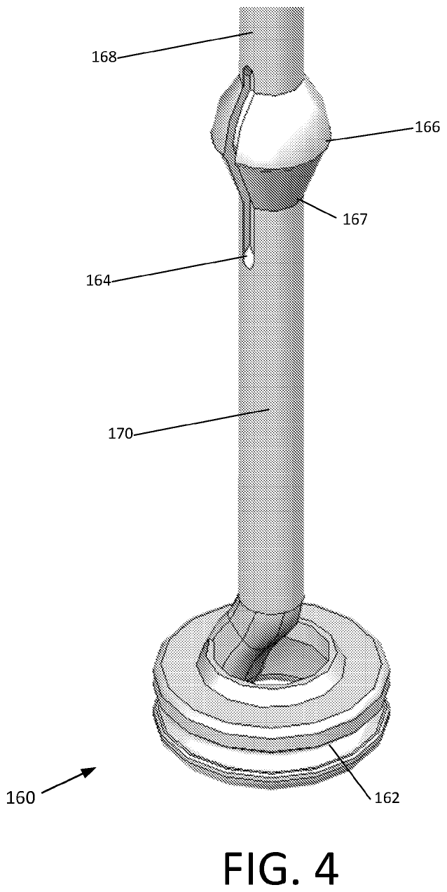

FIG. 4 is a perspective view of a portion of a ring component 160 that may find use with the foreskin holder 100 of FIGS. 1-3. The ring component 160 may include a ring 162, a groove 164, and a bulbous portion 166. The ring 162 may be structurally similar to the “ring 116” of the '918 Patent or ring 716 of FIGS. 7-16 herein, for example, including the associated broader structure of which the ring 162 or ring 716 may be a part. The bulbous portion 166 may be structurally similar to the “position adjustor 134” of the '918 Patent or position adjuster 734 of FIGS. 7-16 herein, for example, including the associated broader structure of which the bulbous portion 166 may be a part. The groove 164 may correspond to a pair of tabs 112 and 114 or a pair of tabs 122 and 124 (with a mirrored groove on the other side of the ring component 160 to correspond with the other pair of tabs-collectively “grooves 164”), such that the grooves 164 are adequately shaped to receive the tabs 112, 114, 122, and 124 and to enable distal (or longitudinal) movement while restricting lateral movement. The bulbous portion 166 may have a spherical or bulbous shape that corresponds with the first concave portion 116 and the second concave portion 126, and the lower section of the bulbous portion 166 may form a lip 167.

The groove 164 may extend longitudinally and may be defined in the bulbous portion and in respective cylindrical portions 168, 170 that are proximal and distal of the bulbous portion 166. The groove 164 may have a uniform radial depth along its length and/or a uniform lateral width along its length, in some embodiments. The radial depth and/or lateral width of the groove 164 may be sufficient to retain a tab 112, 114, 122, 124 during longitudinal movement of the tab 112, 114, 122, 124 and to prevent lateral movement of the tab 112, 114, 122, 124. In some embodiments, a pair of grooves may be provided, with a second groove that is identical to groove 164 mirrored on an opposed side of the ring component 160.

Referring to FIGS. 1-4, the first arm 110 and the second arm 120 may interact with the ring component 160 to enable the foreskin holder 100 to be movable from a first position to a second position. In a first position (e.g., disengaged position), the first arm 110 and second arm 120 may be positioned about the bulbous portion 166 of the ring component 160. The first concave portion 116 and the second concave portion 126 may, in the first position, be flush with or otherwise snug about the bulbous portion 166 to hold the foreskin holder 100 in the first position.

In the second position (e.g., engaged position), the first arm 110 and second arm 120 may disengage from the bulbous portion 166 by sliding the foreskin holder 100 distally (e.g., along an axis formed from the first arm 110 and second arm 120 to the ring portion 150), where “distal” refers to a direction that is towards the patient. The lip 167 may provide a proximal boundary (where “proximal” refers to a direction that is away from the patient) for the foreskin holder 100 when in the second position, such that the lip 167 prevents movement or provides resistance against the foreskin holder 100 returning to the first position from the second position.

When the foreskin holder 100 is in the first position, the ring portion 150 may be displaced from the ring 162 of the ring component 160 that interacts with the ring portion 150 to compress a foreskin. As such, the foreskin holder 100 may be placed in the first position to enable a foreskin to be positioned between the ring portion 150 and the ring 162. In the second position, the ring portion 150 is flush (or substantially flush) with the ring 162 and provides a clamping force in conjunction with the ring 162. As such, the foreskin holder 100 may be placed in the second position once a foreskin is in place to hold that foreskin stationary for the male circumcision process.

As discussed above, the grooves 164 may correspond with tabs 112, 114, 122, and 124, and may run longitudinally along a length of the ring component 160 to align with an axis of movement for the foreskin holder 100 from the first position to the second position. When the foreskin holder 100 moves from the first position to the second position, the tabs 112, 114, 122, and 124 of the foreskin holder 100 may slide along the grooves 164. Because the tabs 112, 114, 122, and 124 are constrained by the grooves 164, the movement of the foreskin holder 100 may be similarly restricted, meaning that while the foreskin holder 100 is moving distally from the first position to the second position, rotation about the ring component 160 is prevented. By improving the lateral stability of the foreskin holder 100, and preventing rotation of the foreskin holder 100 while in the second position (e.g., not just during movement of the foreskin holder 100), the foreskin may be held more securely, leading to more reliable and consistent incisions.

FIGS. 5A and 5B illustrate two states of use of an example foreskin holder 200 and an example ring component 250. FIG. 5A illustrates a disengaged state, and FIG. 5B illustrates an engaged state. The foreskin holder 200 may include a first arm 202, a second arm 204, and a ring portion 206. The ring component 250 may include a bulbous portion 252, a first lip 253, a second lip 254, and a ring 256. The ring portion 206 may be structurally similar to the “ring portion 156” of the '918 Patent or ring portion 756 of FIGS. 7-16 herein, for example, including the associated broader structure of which the ring portion 206 may be a part. The ring 256 may be structurally similar to the “ring 116” of the '918 Patent or ring 716 of FIGS. 7-16 herein, for example, including the associated broader structure of which the ring 256 may be a part.

During a male circumcision, the foreskin is fed through an interior of the ring 256 of the ring component 250 and spread across a top surface of the ring 256. When the foreskin is in place, the foreskin holder 200 is moved distally along the ring component 250 from the disengaged state to the engaged state and the ring portion 206 of the foreskin holder 200 clamps the spread foreskin along with the proximal surface of the ring 256. In the disengaged state, the first arm 202 and the second arm 204 each may be flush with and secured upon the bulbous portion 252. Due to the relatively concave shapes of the first arm 202 and second arm 204 and the relatively convex shape of the bulbous portion 252, the foreskin holder 200 may be held in place by the interaction between the first arm 202, second arm 204, and bulbous portion 252. The first lip 253 may resist proximal movement of the arms 202, 204 when the foreskin holder 200 is moved to the longitudinal position of FIG. 5B and the rotational position of FIG. 5A. The first lip 253 may have a relatively shallow radial depth, and thus sufficient proximal force may allow the arms 202, 204 to flex outward and move over the first lip 253 and the foreskin holder 200 to be moved back to the disengaged state of FIG. 5A.

Once the foreskin holder 200 is moved distally to the distal position of FIG. 5B and the rotational position of FIG. 5A, the foreskin holder 200 may be rotated about the longitudinal axis to the engaged state of FIG. 5B. In the distal position, the foreskin holder 200 may be rotated without damaging the clamped foreskin, as the frictional force generated by the rotation is relatively low. When rotated, the first arm 202 and second arm 204 abut the second lip 254, which functions as a locking mechanism and prevents proximal movement of the foreskin holder 200. The second lip 254 may have a greater radial depth than the first lip 253, and therefore the arms 202, 204 may not be able to flex over the second lip 254, unlike the first lip 253.

Because the second lip 254 prevents proximal longitudinal motion of the foreskin holder 200, the foreskin holder 200 is essentially locked in place, and a stronger and more consistent clamping force may be applied to the foreskin.

FIGS. 6A and 6B illustrate two states of use of an example foreskin holder 300 and an example ring component 350. FIG. 5A illustrates a disengaged state, and FIG. 5B illustrates an engaged state. The foreskin holder 300 may include a first arm 302, a second arm 304, and a ring portion 306. The ring component 350 may include a bulbous portion 352, a first list 353, a second lip 354, and a ring 356. The ring portion 306 may be structurally similar to the “ring portion 156” of the '918 Patent or the ring portion 756 of FIGS. 7-16 herein, for example, including the associated broader structure of which the ring portion 306 may be a part. The ring 356 may be structurally similar to the “ring 116” of the '918 Patent or the ring 716 of FIGS. 7-16 herein, for example, including the associated broader structure of which the ring 356 may be a part.

The foreskin holder 300 and the ring component 350 interact similarly to the foreskin holder 200 and the ring component 250. A primary difference is a shape of the bulbous portion 352 compared to the bulbous portion 252: the former may greater radial depth and may transition to the second lip 354 at a substantially right angle, while the latter may be a more football-shaped curve and may transition more gradually to the second lip 254. Due to the shape of the bulbous portion 352, the benefits provided by the rotatability of the foreskin holder 300 and the protrusion of the second lip 354 may be more drastic or impactful.

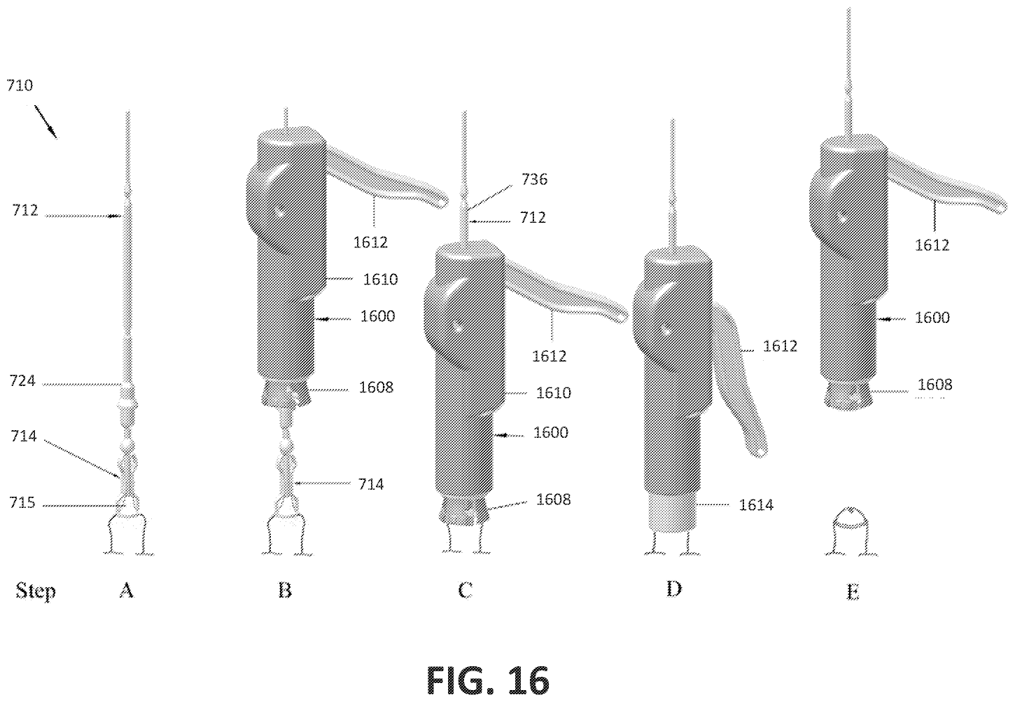

FIGS. 7A and 7B illustrate an example pressure applicator 710 that includes ring component 712 and foreskin holder 714. FIGS. 7A and 7B illustrate ring component 712 and foreskin holder 714 in the disengaged and engaged positions, respectively. Ring component 712 is a single, one-piece, solid member made from, for example, injection molding of a malleable, elastic material (such as plastic) with an open ring 716 mounted orthogonally to lower shaft 718 by curved member 720, which allows manipulation of ring 716 within the foreskin. Ring component 712 includes a position adjustor 734 that delineates the lower shaft 718 from the upper shaft 736. Ring 716 can include an opening or gap 730 large enough to allow the thickness of the foreskin to enter. Ring 716 can include an inner diameter 732 large enough to receive a predetermined sized glans and shield the glans or head of the penis from being clamped and/or cut. Lower Shaft 718 (not shown) and/or upper shaft 736 (shown) can include a plurality of diameters 722A, 722B to control the insertion of ring component 712 into a clamping-cutting device 200 (disclosed in PCT international application PCT/US2005/022404 and incorporated herein by reference). A shaft diameter change can be delineated by a notch or ledge or, as illustrated in FIG. 21A, shoulder 724 that acts as a stop of ring component 712 into clamping-cutting device 200 (see FIG. 16).

Ring 716 includes a holding/cutting surface 726 on its top surface being adapted to act as a holding surface when foreskin holder 714 is engaged and a cutting surface when a cutting edge of a blade of a clamping-cutting device is pressed down against foreskin interposed between holding/cutting surface 726 and the cutting edge. Ring 716 may also include grooved outer surface 728 adapted to engage with inward radial extension or projection 1606 of retractable arm 1608 (see FIG. 16) to hold ring component 712 in a stationary position relative to clamping-cutting device 1600 during the clamping/cutting operation, such that the foreskin 115 is trapped between the grooved outer surface 728 and the radial extension 1606 of the retractable arms 1608. Grooved outer surface 728 will interact with inward radial extension 1606 to crush the foreskin against grooved outer surface 728 and contemporaneously hold ring component 712 in position while circular blade 204 is delivered to make the incision in the foreskin.

Now returning to FIGS. 7A and 7B, one example of the self-adjusting pressure applicator 710 illustrates position adjustor 734 that cooperates with malleable, spring-like opposing arms 738 of foreskin holder 714 to apply a hands-free self-adjusting constant pressure force upon the foreskin disposed between lower surface 740 of foreskin holder 714 and holding/cutting surface 726 of ring component 712. Malleable arms 738 and position adjustor 734 act as pressure force generators. This arrangement provides for flexibility in positioning the foreskin prior to cutting while maintaining a predetermined pressure force. One embodiment for the present invention applies a pressure force between 50 and 200 grams to the tissue when engaged. Another embodiment applies a pressure force of about 181 grams of force to the tissue when engaged. The pressure force is exerted along a common centerline of ring component 712 and foreskin holder 714.

In some embodiments, the self-adjusting pressure applicator 710 can be engaged ranging from about 30 seconds to about one minute, the foreskin can be positioned between the two components, the clamp applied, and the retained foreskin crushed and excised. The agility, flexibility, and simplicity of the self-adjusting pressure applicator 710 allows for repositioning of the foreskin to assure substantially full or entire circumferential retention of the foreskin or tissue. Such minor adjustments can be made with great precision within a short period of time and with no tissue damage. Providing full circumferential retention of the foreskin or tissue along the crushing and cutting surface results in an improvement of the surgical removal of the foreskin or tissue without the use of barbs, pins, clips, or hemostats.

In other embodiments of the self-adjusting pressure applicator 710, pressure is applied indefinitely to the tissue being retained allowing for prolonged surgical manipulation of that tissue without causing permanent injury.

One embodiment of position adjustor 734 is generally a circular body, having a tapered surface, and can be, for example, in the form of two unequal half spheres (similar to football halves) that create a shoulder or lip 742 when joined together. An upper half sphere 744 can have a diameter at the joining interface 748 larger than the mating diameter of lower half sphere 746. Spheres 744, 746 each have a tapered surface to facilitate the upward and downward movement of malleable arms 738 on the spheres 744, 746. The shoulder 742 formed at interface 748 acts as a malleable arm stop to inhibit the upward progress of malleable arms 738 as the foreskin is pulled upward to position it for crushing and cutting. The malleable arms 738 must be spread open to fit over shoulder 742 and into the disengaged position. The concave configuration of malleable arms 738 can be sized and shaped similar to spheres 744, 746 for containment of the foreskin holder 714 while in the disengaged position (FIG. 7A) and for spring-back properties while in the engaged position (FIG. 7B). The tapered surface of lower sphere 746 can be sized and shaped to provide the desired predetermined pressure force taking into consideration the spring-back force of malleable arms 738. Malleable arms 738 must always be in contact with lower sphere 746 to maintain a constant predetermined pressure on to the foreskin when foreskin is disposed between lower surface 740 of foreskin holder 714 and holding/cutting surface 726 of the ring component 712.

To hold foreskin in a precise location, when the foreskin holder 714 is positioned in the down or engaged position, malleable arms 738 of the foreskin holder 714 interact with the lower tapered surface 746 of position adjustor 734 forming a spring-like mechanism to create a force in the direction towards holding/cutting surface 716 and away from position adjustor 734. The spring-like mechanism exerts a force that holds foreskin holder 714 in place along the holding/cutting surface 726 of ring component 712. The pressure force in turn holds the foreskin in place that is positioned between the holding/cutting surface 726 and the foreskin holder ring portion 756. Ring portion 756 at the base of the foreskin holder 714 applies a 360 degree circumferential force to the holding/cutting surface 726 to achieve a substantially constant circumferential pressure or holding force. Shoulder 742 of the position adjustor 734 along interface 748 acts as a latch allowing for malleable arms 738 to move down lower shaft 718, but prevents inadvertent movement of foreskin holder 714 back up into the disengaged position. Shoulder 742 ensures that malleable arms 738 and lower tapered surface 746 maintain in an axial position that generates the spring-like force holding the foreskin holder 714 in place relative to holding/cutting surface 726 of ring component 712. Ring portion 756 of foreskin holder 714 is preferably supported by two support arms 158 (discussed in detail below) that allow for easy visibility and access to the foreskin that becomes retained within the cavity 161 of foreskin holder 714.

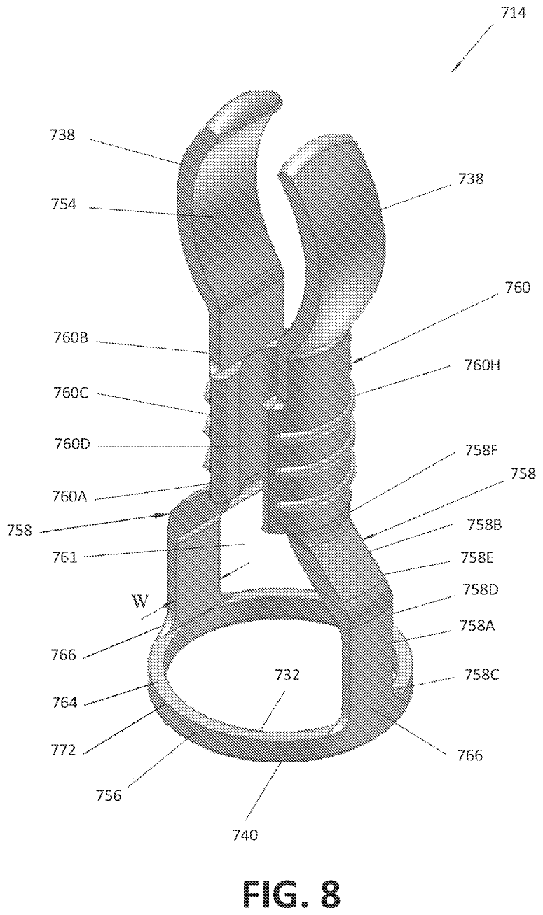

Now turning to FIG. 8, one example of the foreskin holder 714 is a single, one-piece, injected molded, solid member constructed of malleable, elastic material (such as plastic). As mentioned above, foreskin holder 714 includes a ring portion 756, a pair of support arms 758, a C-shaped sleeve 760, and a pair of malleable arms 738. Ring portion 756 is configured to align juxtaposition to the holding/cutting surface 726 of ring 716 (see FIG. 7A) of the ring component 712. Ring portion 756 is generally circular with an inner diameter 732, outer diameter 172, a bottom surface 740, and a top surface 764. Bottom surface 740 is a pressure surface that contacts and applies pressure to the foreskin disposed between the holding/cutting surface 726 of the ring component 712.

As discussed above, malleable arms 738 will exert a downward force caused by its interaction with a position adjustor 734 on the modified ring component 712. The spring coefficient of the malleable arms 738 can be derived by well known methods to accommodate the desired pressure force. The desired pressure force can be determined by knowing, among other characteristics, thickness of tissues including but not limited to foreskin, brain or neuron tissue, or cardiac vessel. The downward force is translated through C-shaped sleeve 760 to support arms 158. Supports arms 158 applies the translated force to ring portion 756 at interconnect points 766. The force will be substantially evenly distributed along lower surface 740 when lower surface 740 is in contact with foreskin disposed between lower surface 740 and holding/cutting surface 726 of the ring component 712. The force or contact pressure distribution along to lower surface 740 is a function of width W of support arms 158 and the rigidity of ring portion 756. For example, the wider the support arms 158, the more ring portion surface area in the direct load path of the translated force. Ring portion 756 would bend less when the ring portion 756 is more rigid. Bending of ring portion 756 could cause a reduction of the contact pressure along lower surface 740 as a function of circumferential distance from the interconnect points 766.

Though a pair of independently deflectable malleable arms have been used to illustrate a spring-like mechanism to apply a pressure force, it should be appreciated that there are many suitable combinations of arm (deflectable or rigid) or spring-like mechanisms incorporated into the body of the position adjustor 734 that will achieve the desired results. The invention should not be limited to only the embodiments disclosed in this application.

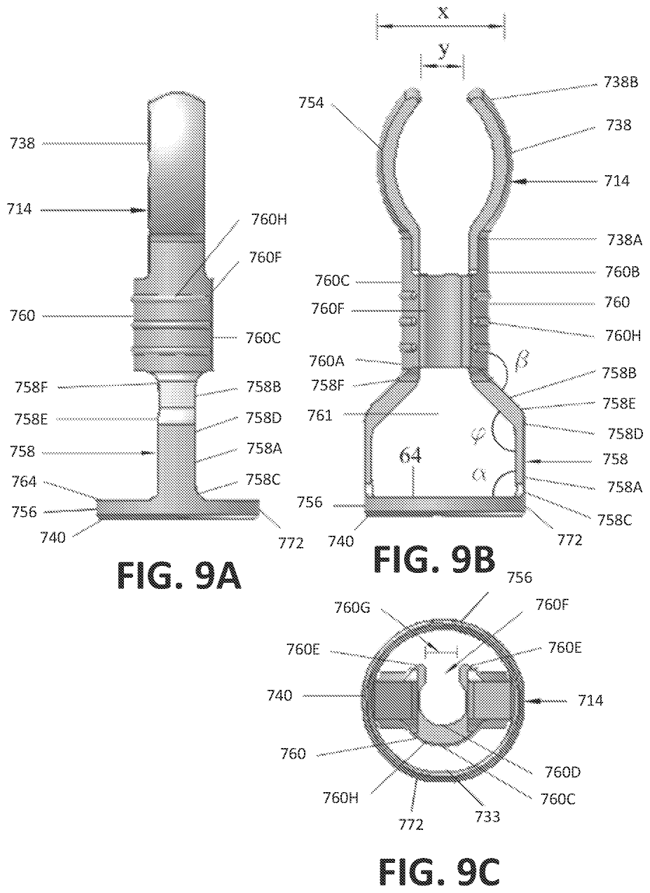

Now turning in FIGS. 9A-C, one embodiment of a support arm 158 can include one or more sections. Shown as an example is support arm 158 having two sections 158A, 158B. Two support arms 158 provides for easier visibility and access to the foreskin that becomes retained within the foreskin holder 714. Section 158A is attached at one of its ends 158C to either top surface 764 or to the inner diameter 733 (FIG. 23C) or to the outer diameter 172. One embodiment of Section 158A can be oriented orthogonal to ring portion 756. However any angle α (FIG. 9B) that applies evenly distributed forces along lower surface 740 of ring portion 756 is acceptable. Section 158B is attached at a predetermined angle β at one of its ends 158E to the other end 158D of section 158A, and at its other end 158F to C-shaped sleeve 760 at its end 760A at a predetermined angle β. The angular relationships between sections 158A and 158B and C-shaped sleeve 760 create an inward taper of support arms 158. The predetermined angles α, φ, and β, and therefore the support arm taper, can be variable to accommodate the independent optimization of ring portion 756 and C-shaped sleeve 760, and/or to optimize the contact pressure along lower surface 740. In the case where φ is 180 degrees, there will only be one section 158. Though the two support arms are illustrated and described herein, any appropriate number of support arms may be included.

Each malleable arm 738 is attached at one of its ends 738A to end 760B of C-shaped sleeve 760 and its other end 738B is free to independently flex or deflect (X) and form a gap (Y) at rest. Free ends 738B can deflect to predetermined distance X under predetermined load or force conditions (discussed in detail below). The flexed or deflected malleable arms act like a spring storing energy to produce a spring back force, which is a function of the deflection distance X. The larger the deflection distance X, the more spring-back force will be created. Since the spring back force is also a function of the materials property, for example the modulus of elasticity, material selection is also important in determining the desired spring back force. It will become apparent later in this application that the spring back force will act upon a position adjustor 734 of ring component 712 to result in a downward movement of foreskin holder 714 that applies the pressure force to the foreskin disposed between lower surface 740 and holding/cutting surface 726 of ring component 712. Malleable arms 738 can be of any configuration that results in the desired spring back force working in conjunction with position adjustor 734 of ring component 712. For example, malleable arms 738 can have a concave surface 754 and be opposingly oriented to each other. As discussed above, the type of materials, dimensions (thickness, width, length) of malleable arms 738, and/or shape of malleable arms 738 (concave or convex) are derivable from the desired pressure force to be applied to a target tissue, such as foreskin, brain or neuro tissue, or cardiac vessel.

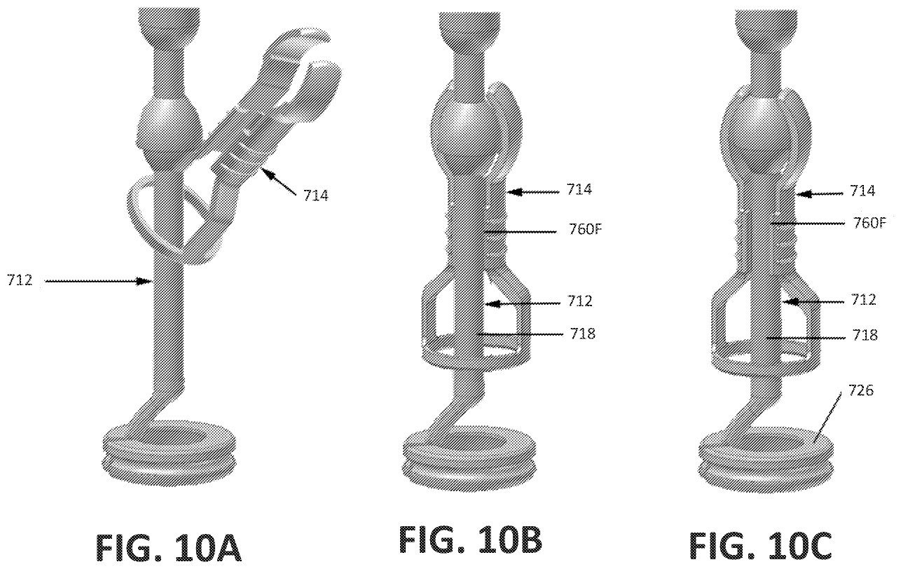

Now turning to FIG. 9C, C-shaped sleeve 760 in generally cylindrical in shape and hollow having an outer diameter 760C, an inner diameter 760D, and two circumferential ends 760E, which form slot 760F with gap 760G. Gap 760G is less than inner diameter 760D of C-shaped sleeve 760 and less than the outer diameter 176 of lower shaft 718 of ring component 712 (FIG. 7A). C-shape sleeve 760 has sufficient elastic properties such that gap 760G will open as lower shaft 718 is inserted through slot 760F and gap 760G will close or return to its original gap distance 760G after insertion of lower shaft 718 into slot 760F. Once lower shaft 718 is inserted through slot 760F, it is trapped or entrained within C-shaped sleeve 760 and only permitted to move along in a longitudinal path within C-shape sleeve 760 because outer diameter 176 is smaller than inner diameter 760D to allow for lower shaft 718 to move longitudinally within inner diameter 760D. Ends 760E can be rounded to facilitate easier insertion of lower shaft 718 through slot 760F. FIGS. 10A-C illustrate foreskin holder 714 (female component) being positioned on ring component 712 (male component) (FIG. 10A), alignment of slot 760F of foreskin holder 714 with lower shaft 718 of ring component 712 prior to insertion (FIG. 10B), and foreskin holder 714 slidably connected with ring component 712 after insertion of lower shaft 718 through slot 760F (FIG. 10C).

One embodiment of outer diameter 760C can include finger grips 760H to facilitate a better grip for raising and lowering foreskin holder 714 relative to ring component 712. Finger grips 760H can be one or more projections. As shown in FIGS. 9A and 9B, three projections 760H are circumferentially oriented parallel to each other along outer diameter 760C. Any cluster or grouping of projections are acceptable as well as any surface treatment that creates a frictional condition between the user's fingers and outer diameter 760C.

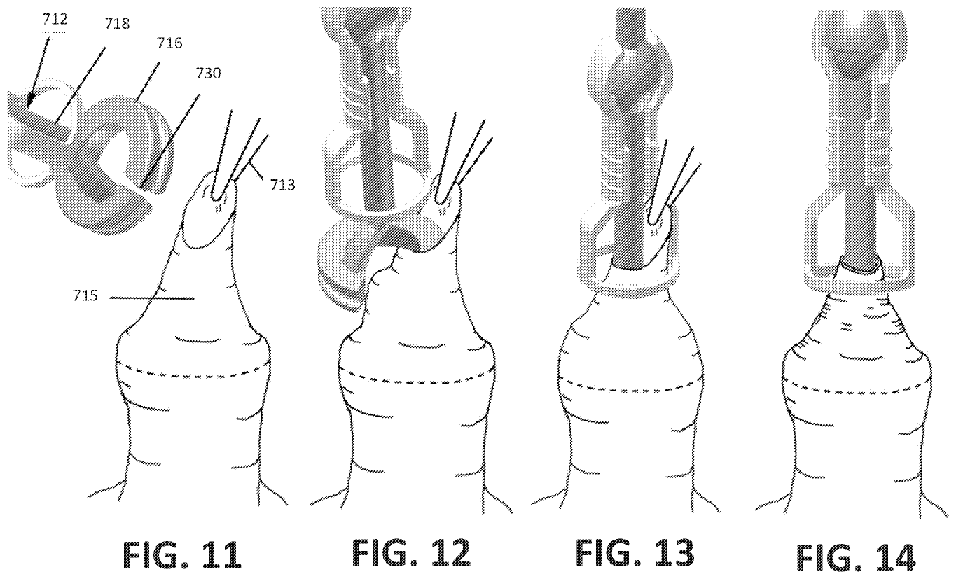

FIGS. 11-14 demonstrate an example of the insertion of ring 716 into the foreskin 115. As discussed above, gap 730 allows entry of ring 716 into foreskin 115. Foreskin 115 is held by an atraumatic forceps 713 while gap 730 is positioned to enter foreskin 715. With a pair of non-traumatic forceps, the foreskin is gently grasped and foreskin 715 is guided into the gap 730 of ring 716. With a screw-like motion of lower shaft 718 of ring component 712, the ring 716 is advanced in, down, and around the inner aspect of the foreskin 715. The gap 730 in ring 716 allows the ring 716 to be advanced into the foreskin with a smooth, non traumatic fluid screwing motion. Once ring 716 is fully inserted and resides just beneath foreskin 715, it can be pushed down slowly toward the glans to free any adhesions but to be surrounded by foreskin 715. Inside foreskin 715, gap 730 is closed by the elastic nature of the foreskin 715 attempting to return to its state after being stretched during the insertion of ring 716. The closed ring 716, inside the foreskin of the penis, residing just above the glans or tip of the penis, is then used as the compressive surface for any number of clamps, such as an inward radial extension (tongue) and mating recess, and its top surface 726 acts as a combination glans shield, cutting surface for the blade as it cuts the foreskin, and foreskin holder when cooperating with foreskin holder 714.

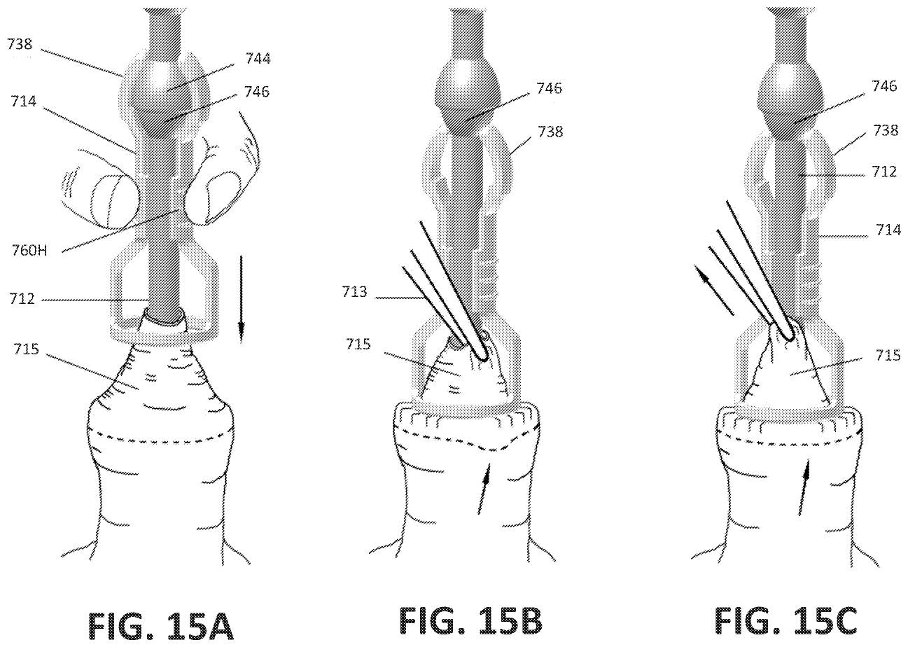

FIGS. 15A-C illustrate the operation of foreskin holder 714 after foreskin is positioned on the ring 716 of ring component 712 (hidden beneath foreskin 715). The operator pinches or grips finger grips 760H of foreskin holder 714 and pushes foreskin holder 714 down towards holding/cutting surface 726 (hidden beneath foreskin 715) (FIG. 15A). The dashed line represents the desired circular foreskin cutting circumference. Malleable arms 738 automatically open as malleable arms 738 move down upper sphere 744 and then automatically close as malleable arms 738 transition to move down lower sphere 746. The operator uses forceps 713 to grab the foreskin 715 for initial positioning for a substantially circular cut, shown as a dashed line (FIG. 15B). After initial positioning, the operator pulls the foreskin 715 substantially upwards (FIG. 15C). Foreskin holder 714 may advance upward on to lower sphere 746. As foreskin holder 714 advances upward the spring back force of malleable arms 738 increases and the downward pressure increases to counter the upward pull on the foreskin and to maintain a substantially constant circumferential pressure force onto the foreskin. Repositioning of the forceps may be necessary to assure the foreskin is evenly distributed around holding/cutting surface 726 of ring component 712 (hidden beneath foreskin 715) for a substantially circular cut (dashed line).

FIG. 16 illustrates one embodiment of the self-adjusting pressure applicator 710 adapted for use with a circumcision clamping-cutting device 1600 (disclosed in PCT international application PCT/US2005/022404 and incorporated herein by reference) adapted to cooperate with each other. Step A: Ring 716 (hidden beneath foreskin) of ring component 712 is inserted into the foreskin 715 of the penis, as discussed above. Step B: Clamping-cutting device 1600 is set in the open position with lever arm 1612 rotated up and retractable arms 1608 extending fully through the bottom of housing 1610 and sleeve 1614. Step C: Clamping-cutting device 1600 is inserted onto upper shaft 736 of ring component 712. Downward movement of clamping-cutting device 1600 and housing 1610 onto upper shaft 736 is stopped when shoulder 724 contacts bottom face of an internal thru hole (not shown). Step D: Lever arm 1612 is activated downward to advance sleeve 1614 downward over retractable arms 1608 causing the clamping force and delivering an internal circular blade to the foreskin 715. Sleeve 1614 moves downwardly over retractable arms 1608 causing retractable arms 1608 to radially close on to and to exert lateral compressive force against the foreskin 715. Retractable arms 1608 exert sufficient lateral compressive force or clamping such that inward radial extensions of retractable arms 1608 forces the foreskin 715 into grooved outer surface 728 of ring 716, thereby clamping the foreskin 715. As sleeve 1614 is advanced even further over the ring 716, the blade is delivered to the top surface 764 of ring 716 and creates the circular incision into the foreskin 715 when ring 716 is positioned within clamping-cutting device 1600. Using the top surface 764 of ring 716 as the cutting surface that is held in place by the closed retractable arms 1608, the blade makes a single, clean, circumferential incision on top surface 764 of ring 716, removing the excess foreskin 715. The clamp is left in place for a period of time ensuring adequate crushing and hemostasis. Step E (FIG. 16): Lever arm 1612 is lifted upward and retractable arms 1608 release ring component 712, lifting sleeve 1614 of the clamping-cutting device 1600 and lifting the blade back up into housing 1610 and releasing ring 716. Ring component 712 with severed foreskin is permanently locked within housing to prevent inadvertent reuse of any of the components of the device. All components and byproducts of the operation are thrown away, thereby completing the circumcision.

In a first aspect of the present disclosure, a device for performing a male circumcision is provided. The device includes a ring component that includes a first ring portion anda shaft defining a longitudinal axis and extending proximally from the ring portion, wherein the shaft comprises a longitudinal groove. The device further includes a foreskin holder coupled to the ring component and configured to move axially along the shaft, the foreskin holder including a lateral tab, extending radially inward, that engages with the longitudinal groove to inhibit lateral movement of the foreskin holder with respect to the ring component, and a second ring portion, wherein distal movement of the second ring portion clamps a foreskin between the first ring portion and the second ring portion.

In an embodiment of the first aspect, the foreskin holder includes two axially-aligned lateral tabs, both configured to engage with the longitudinal groove.

In an embodiment of the first aspect, the shaft includes two longitudinal grooves and the foreskin holder includes two lateral tabs, each of which engages with a respective one of the two longitudinal grooves. In a further embodiment of the first aspect, the foreskin holder comprises four lateral tabs comprising two pairs of axially-aligned lateral tabs, each pair of axially-aligned lateral tabs engaging with a respective one of the two longitudinal grooves. In a further embodiment of the first aspect, the foreskin holder comprises two arms, each comprising a concave portion, wherein each pair of tabs is associated with a respective one of the arms, with a first one of the tabs in the pair disposed on a longitudinal top of the concave portion and a second one of the tabs in the pair disposed on a longitudinal bottom of the concave portion. In a further embodiment of the first aspect, the two longitudinal grooves are radially opposed from each other.

In an embodiment of the first aspect, the shaft comprises a bulbous portion, wherein the longitudinal groove extends into the bulbous portion. In a further embodiment of the first aspect, the foreskin holder comprises an arm disposed proximally of the second ring portion, the arm comprising a concave portion that interacts with the bulbous portion of the shaft, the lateral tab defined on the arm.

In an embodiment of the first aspect, the longitudinal groove has a uniform radial depth along a longitudinal length of the groove with respect to an outer surface of the shaft.

In an embodiment of the first aspect, the lateral tab is disposed on a longitudinal top or bottom of a concave portion of an arm of the foreskin holder.

In a second aspect of the present disclosure, a device for performing a male circumcision is provided that includes a ring component including a first ring portion and a shaft defining a longitudinal axis and extending proximally from the ring portion, wherein the shaft comprises a laterally-projecting lip, and a foreskin holder coupled to the ring component and configured to move axially along the shaft, the foreskin holder including an arm that engages with the laterally-extending lip to lock the axial position of the foreskin holder, and a second ring portion, wherein distal movement of the second ring portion clamps a foreskin between the first ring portion and the second ring portion.

In an embodiment of the second aspect, the shaft includes two radially-opposed laterally-extending lips. In a further embodiment of the second aspect, the foreskin holder comprises two radially-opposed arms, each of which arms engages with a respective one of the two radially-opposed lips.

In an embodiment of the second aspect, the lip is at a distal end of a bulbous portion of the shaft, and the arm includes a concave portion terminating in a distal tip, wherein the concave portion rests on the bulbous portion of the shaft in a first state and the distal tip engages with the lip in a second state. In a further embodiment of the second aspect, the arm is moved from the first state to the second state by an axially distal movement of the arm followed by a rotation of the foreskin holder about the axis. In a further embodiment of the second aspect, the lip is a primary lip, and wherein the bulbous portion comprises a secondary lip that is circumferentially offset from the primary lip, with respect to the longitudinal axis, by ninety degrees. In a further embodiment of the second aspect, the secondary lip has a radial depth that is less than a radial depth of the primary lip. In a further embodiment of the second aspect, the shaft comprises two radially-opposed laterally-extending primary lips and two radially-opposed laterally-extending secondary lips.

In an embodiment of the second aspect, the lip has a radially arcuate radially outermost surface.

In an embodiment of the second aspect, the lip has a radially flat radially outermost surface.

While this disclosure has described certain embodiments, it will be understood that the claims are not intended to be limited to these embodiments except as explicitly recited in the claims. On the contrary, the instant disclosure is intended to cover alternatives, modifications and equivalents, which may be included within the spirit and scope of the disclosure. Furthermore, in the detailed description of the present disclosure, numerous specific details are set forth in order to provide a thorough understanding of the disclosed embodiments. However, it will be obvious to one of ordinary skill in the art that systems and methods consistent with this disclosure may be practiced without these specific details. In other instances, well known methods, procedures, components, and circuits have not been described in detail as not to unnecessarily obscure various aspects of the present disclosure.

What is claimed is:

1. A device for performing a male circumcision, the device comprising:
a ring component comprising:
a first ring portion; and
a shaft defining a longitudinal axis and extending proximally from the ring portion, wherein the shaft comprises a longitudinal groove; and
a foreskin holder coupled to the ring component and configured to move axially along the shaft, the foreskin holder comprising:
a lateral tab, extending radially inward, that engages with the longitudinal groove to inhibit lateral movement of the foreskin holder with respect to the ring component; and
a second ring portion, wherein distal movement of the second ring portion clamps a foreskin between the first ring portion and the second ring portion.

2. The device of claim 1, wherein the foreskin holder includes two axially-aligned lateral tabs, both configured to engage with the longitudinal groove.

3. The device of claim 1, wherein the shaft comprises two longitudinal grooves and the foreskin holder includes two lateral tabs, each of which engages with a respective one of the two longitudinal grooves.

4. The device of claim 3, wherein the foreskin holder comprises four lateral tabs comprising two pairs of axially-aligned lateral tabs, each pair of axially-aligned lateral tabs engaging with a respective one of the two longitudinal grooves.

5. The device of claim 4, wherein the foreskin holder comprises two arms, each comprising a concave portion, wherein each pair of tabs is associated with a respective one of the arms, with a first one of the tabs in the pair disposed on a longitudinal top of the concave portion and a second one of the tabs in the pair disposed on a longitudinal bottom of the concave portion.

6. The device of claim 3, wherein the two longitudinal grooves are radially opposed from each other.

7. The device of claim 1, wherein the shaft comprises a bulbous portion, wherein the longitudinal groove extends into the bulbous portion.

8. The device of claim 7, wherein the foreskin holder comprises an arm disposed proximally of the second ring portion, the arm comprising a concave portion that interacts with the bulbous portion of the shaft, the lateral tab defined on the arm.

9. The device of claim 1, wherein the longitudinal groove has a uniform radial depth along a longitudinal length of the groove with respect to an outer surface of the shaft.

10. The device of claim 1, wherein the lateral tab is disposed on a longitudinal top or bottom of a concave portion of an arm of the foreskin holder.

11. A device for performing a male circumcision, the device comprising:
a ring component comprising:
a first ring portion; and
a shaft defining a longitudinal axis and extending proximally from the ring portion, wherein the shaft comprises a laterally-projecting lip; and
a foreskin holder coupled to the ring component and configured to move axially along the shaft, the foreskin holder comprising:
an arm that engages with the laterally-extending lip to lock the axial position of the foreskin holder; and
a second ring portion, wherein distal movement of the second ring portion clamps a foreskin between the first ring portion and the second ring portion.

12. The device of claim 11, wherein the shaft comprises two radially-opposed laterally-extending lips.

13. The device of claim 12, wherein the foreskin holder comprises two radially-opposed arms, each of which arms engages with a respective one of the two radially-opposed lips.

14. The device of claim 11, wherein:
the lip is at a distal end of a bulbous portion of the shaft; and
the arm comprises a concave portion terminating in a distal tip, wherein the concave portion rests on the bulbous portion of the shaft in a first state and the distal tip engages with the lip in a second state.

15. The device of claim 14, wherein the arm is moved from the first state to the second state by an axially distal movement of the arm followed by a rotation of the foreskin holder about the axis.

16. The device of claim 14, wherein the lip is a primary lip, and wherein the bulbous portion comprises a secondary lip that is circumferentially offset from the primary lip, with respect to the longitudinal axis, by ninety degrees.

17. The device of claim 16, wherein the secondary lip has a radial depth that is less than a radial depth of the primary lip.

18. The device of claim 16, wherein the shaft comprises two radially-opposed laterally-extending primary lips and two radially-opposed laterally-extending secondary lips.

19. The device of claim 11, wherein the lip has a radially arcuate radially outermost surface.

20. The device of claim 11, wherein the lip has a radially flat radially outermost surface.
